(19) United States
(12) Patent Application Publication
(10) Pub. No.: US 2022/0415486 A1
(43) Pub. Date: Dec. 29, 2022

(54) DATA GATHERING AND MANAGEMENT FOR HUMAN-BASED STUDIES

(71) Applicant: International Business Machines Corporation, Armonk, NY (US)

(72) Inventors: Zachary Shahn, Brooklyn, NY (US); Uri Kartoun, Cambridge, MA (US); Daby Mousse Sow, Croton on Hudson, NY (US); Kenney Ng, Arlington, MA (US); Jianying Hu, Bronx, NY (US)

(21) Appl. No.: 17/362,862

(22) Filed: Jun. 29, 2021

(51) Int. Cl. G16H 40/20; G16H 10/20; G16H 50/70; G16H 50/20; G16H 10/60; G16H 15/00

(52) U.S. Cl. CPC G16H 40/20; G16H 10/20; G16H 50/70; G16H 50/20; G16H 10/60; G16H 15/00; A61B 5/48

(57) ABSTRACT

A request to execute a sequentially randomized controlled trial (sRCT) that relates to a subject regarding a population of humans is received. Datapoints from the population that are needed for the sRCT and factors that define the population as fitting the sRCT are identified. It is detected that a human within an interaction satisfies the factors and therefore is part of the population. During the interaction, the datapoints from the human that are needed for the sRCT are gathered in response to detecting that the human is part of the population, and treatment is randomly assigned if the interaction is also a treatment decision point.

### BACKGROUND

Computers are regularly used to organize data (such as health data) for statistical analysis. For example, computers can be used to gather data for randomized controlled trials (RCTs). Once organized, computer software often has specialized abilities to statistically analyze the data, and/or present or organize the data to highlight various findings that could be used to change an end point of that date (e.g., to improve care patients, where the data is health data).

### SUMMARY

Aspects of the present invention relate to a method, system, and computer program product relating to data gathering, data management, and experimental treatment assignment for prospective studies relating to humans. For example, the method includes receiving a request to execute a sequentially randomized controlled trial (sRCT) or sRCT emulation that relates to a subject regarding a population of humans. The method further includes identifying datapoints from the population that are needed for the sRCT or sRCT emulation and factors that define the population as fitting the sRCT. The method further includes detecting that a human within an interaction satisfies the factors and therefore is part of the population. The method further includes gathering, during the interaction, the datapoints from the human that are needed for the sRCT in response to detecting that the human is part of the population.

Beyond this, to describe the method in the context of a medical realm, the method may include randomizing treatment assignments at multiple sequential decision points for each human (e.g., patient) for the sRCT study. A decision point is defined as a point in time at which a treatment action should begin, or a point at which a decision should be made regarding whether to move forward with or otherwise select or change one or more treatment actions. Decision points are possibly conditional on observed history up to each decision point, to enable unbiased estimation of effects of dynamic and time-varying treatment strategies. Dynamic treatment strategies are treatment rules that assign treatments at each treatment decision point based on time-varying characteristics of patients up to that point. The method further includes identifying time points or patient interactions at which it is determined valuable (e.g., preventive, critical) to collect data or assign treatment during execution of the sRCT and evaluate factors that define whether patients are eligible to participate in the sRCT. The method further includes gathering the datapoints from the human that are needed for the sRCT or randomly assigning treatment according to the sRCT protocol in response to detecting that an interaction is relevant to the sRCT. A system and computer product configured to perform the above methods are also disclosed. If sufficient data is collected at each treatment decision point, even if treatment is not randomly assigned but given according to usual care or natural course, it is possible through statistical adjustment to obtain estimates of the same quantities as if the data had been produced by a sRCT that randomly assigned treatment conditional on observed covariates. Thus, the system also includes collection of observational data at each treatment decision point required to estimate treatment effects.

The above summary is not intended to describe each illustrated embodiment or every implementation of the present invention.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a conceptual diagram of an example system in which a controller may gather and manage data for studies relating to humans.

FIG. 2 depicts a conceptual box diagram of example components of the controller of FIG. 1.

FIG. 3 depicts an example flowchart by which the controller of FIG. 1 may gather and manage data for human studies.

FIG. 4 illustrates a cloud environment.

FIG. 5 illustrates a set of functional abstraction layers provided by cloud computing environment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to gathering and managing data and experimentally assigning treatments for prospective studies of human subjects, while more particular aspects of the present disclosure relate to receiving parameters of a sequentially randomized controlled trial (sRCT) and then dynamically and in real-time detecting that humans or interactions should be included in the cohort helpful/required to emulate the sRCT and gathering data on and assigning treatments to these humans within these interactions to complete the sRCT. While the present invention is not necessarily limited to such applications, various aspects of the disclosure may be appreciated through a discussion of various examples using this context.

Randomized trials are the gold standard for studying causal effects of interventions in humans, and sequentially randomized trials are the gold standard for studying effects of interventions delivered at multiple times (referred often as “decision points”). Interventions may include medical treatment strategies, educational techniques, or the like. Computing systems are often configured to gather data and analyze data for such studies. Though sRCTs are predominantly discussed throughout for the purposes of clarity, it is to be understood that this invention is not limited to sRCTs, but that rather any study methodology that focuses on gathering data of subsets of humans in a way that is statistically representative of a greater population of humans for the purpose of identifying statistical trends is consistent with this disclosure (e.g., for example aspects of this disclosure would be understood by one of ordinary skill in the art to relate to randomized controlled trials (RCTs) that are not sequenced).

Aspects of this invention are related to detecting interactions with humans that are relevant to an ongoing sRCT (e.g., identifying treatment decision points or times at which outcomes should be assessed), gathering data from these humans that would be helpful/needed to conduct the sRCT (e.g., information that determines treatment assignments such as current or historical conditions), and prompting random assignments (e.g., medical treatment assignments when the sRCT is a medical sRCT). A computing device that includes a processing unit executing instructions stored on a memory may provide this functionality, this computing device herein referred to as a controller. For example, where an sRCT relates to an ongoing medical treatment study, the controller may be incorporated into one or more medical record keeping systems, such that the controller may analyze medical records as they are received in real-time to identify potential patients and shape visits and/or treatments for the sRCT.

In addition to being configured to assist in gathering information for the sRCT, the controller may be configured to impact interactions with humans to ensure that the sRCT gathers all datapoints required to assess treatment strategies of interest with a sufficiently high confidence score. The primary way the controller might impact interactions is to randomly assign actions such as treatments. For another example, the controller may have access to definitions or itself identify definitions (e.g., by analyzing a corpus of relevant historical records) as to what constitutes a final interaction marking end of follow-up for each human, and/or a final one or more outcomes for the study, so that the sRCT may be concluded and/or so that the controller stops gathering data for that human. At this interaction, the controller may prompt certain variables that may affect the outcome to be collected beyond what be collected in the normal course of treatment, e.g. the controller might prompt a physician to query a patient about aspects of their health relevant to the study but not routinely collected as part of their care. As yet another example, the controller might prompt collection of data that is relevant for conditional random treatment assignment rules within the study but not typically collected during usual care. Beyond this, as discussed herein, the controller may be configured to be sufficiently flexible to react to unexpected data provided by humans, such that datapoints that were not expected are still stored in a manner sufficient for study. In some examples, as provided herein, the controller may further analyze some or all data that is gathered (whether expected or unexpected) to determine if different data needs to be gathered (e.g., by suggesting new or different actions to or for the humans that may enable this data to be generated and therein gathered).

In some examples, the controller may gain approval from the patient before gathering data and/or suggesting treatments (e.g., by prompting a doctor to inform the patient that, being as the patient has condition ABC, would the patient be interested in providing data to the controller and/or follow various treatment programs suggested by the controller to improve knowledge and treatment of condition ABC). Controller 110 may manage treatment strategies by defining treatment actions (e.g., medical procedures, pharmaceutical options, physical rehabilitation) as well as evaluating criteria for what constitutes a treatment decision point). An example of a treatment strategy is to take pharmaceutical A as long as patient has not reported shortness of breath, where if patient experiences shortness of breath, the patient should switch to pharmaceutical B within T months, and in either case the patient should have medical appointments (e.g., at least once per quarter or a year). Though most examples used herein refer to medical studies for the purposes of illustration, it is to be understood that any subject for which human interactions occur and human data in a manner that is consistent with this disclosure is contemplated herein.

The controller may start by receiving a request to conduct an sRCT, where this request includes a subject (e.g., a medical condition a financial analysis for a specific form of investment, a liability analysis for a specific human behavior, or the like) and/or factors that define a population of humans as “fitting” the sRCT (e.g., age, gender, primary medical condition, medical history, socioeconomic characteristics, or the like). In some examples, the controller may also receive information on various confounding variables that are currently known at the start of the study. In certain examples, the controller may identify some of this information during this instantiation phase (e.g., by analyzing a corpus of historical data to identify potential confounding variables). In certain examples, where gathering this data has an associated cost (e.g., for a medical study, this cost may be to the medical professional, patient, or other organization in terms of needed time or resources), the controller may calculate and provide this cost.

Once the test is started, the controller analyzes some or all humans of an organization or network during interactions to determine whether or not they satisfy the factors and therefore are part of the population for the sRCT. For example, where the study is a medical study, the controller may analyze some or all data of current and/or upcoming patient appointments to see if any fit the population. In some examples the controller may flag a patient ahead of time as fitting the population, whereas in other examples the controller may flag the patient as fitting the population in response to data gathered by a medical professional and provided to a medical records system during an appointment. In some examples, the controller may flag patients as potentially included within a population (e.g., based on their health records) and prompt a medical professional to gather specific information about additional factors to verify whether or not the patients are part of the population. For example, the medical professional may ask a specific question that would not typically be included in the patient's electronic health record (EHR), such as whether the patient lives above the 3^{rd} floor in a building with no elevator.

In some examples, the controller may further verify that the population is balanced, such that each aspect of the population is well represented. For example, the population may include people that have a medical condition, and within that the controller may verify that the population is well represented across age, gender, and/or other medical, lifestyle, and demographic factors. In some examples, the controller may flag a human as part of a population that is already well represented, and as such may not gather data from this human as part of the sRCT (or at least not include gathered data from this human into final conclusion, but instead as part of a greater appendix of accompanying information).

Once the controller detects the patient as satisfying the factors and therefore being a part of the population, the controller may gather datapoints from the patient that are needed for the sRCT. For example, the controller may prompt the medical professional to ask for (and/or measure) data of the patient, such as asking lifestyle questions (e.g., do you have an office job that has you sitting most of the day) or gathering physiological data (e.g., getting blood pressure or a cholesterol level). The controller may gather all of this data in a format that is amenable to analysis (e.g., corresponding all responses from and data of the human with all available confounding variables within a table or comma separate value (csv) file or the like).

The controller may further detail various ongoing plans for the human in the future in order to conduct the sRCT. For example, if this is a medical study, the ongoing plan may include specific treatment plans, follow-up appointments, or the like. The controller may further identify each area for which there is a regulatory and/or ethical point for additional consent needed to ask for additional information and/or provide a treatment as part of the sRCT, and prompt the medical professional to obtain such consent at each such point.

While randomized trials are the gold standard for estimating causal effects, it is sometimes unfeasible or unethical to randomize treatment assignments. In such instances, an alternative to a randomized trial is to conduct an observational study that is attempting to emulate an sRCT. Such an observational study is referred to as an sRCT emulation.

However, observational studies often have difficulty with unobserved confounding variables, i.e. variables that differ systematically across groups following different treatment strategies that are also associated with outcomes of interest and thus can lead to differences in outcomes between treatment groups that are not due to treatment received. For example, in a medical study it may be true that if a human has characteristic X, then he or she is more likely to receive treatment A than treatment B and also more likely to experience outcome Y (e.g., such that characteristic X is a confounding variable that, if not appropriately weighed, may cause a study comparing the effect on Y of receiving treatment A versus treatment B to have inaccurate results).

A conventional analysis system may have access to historical data that has been collected for reasons other than the observational study, and as such frequently contains insufficient information on confounding variables that would be needed for causal inference. For example, cholesterol level may be a confounding variable of a medical study, but the historical records may not include cholesterol level so that it cannot be adjusted for. Even if the conventional system (and/or a researcher using the conventional system) identifies all confounding variables, it is often difficult if not impossible for conventional data analysis systems to gain access to sufficient data to provide results that are not biased due to lack of data on confounding variables.

Aspects of this invention may solve or otherwise address these problems of conventional analysis systems. The controller can collect information on relevant confounding variables at all interactions that it identifies as treatment decision points. If a particular confounding variable is not routinely recorded, the controller can prompt the physician to collect the information, e.g. by asking the patient a question or ordering an additional test (e.g., a laboratory value, a vital sign), less commonly available details such as the components of Townsend's deprivation index, and even more comprehensive observations such as a genetic profile or data gathered from wearable devices.

In some examples, the controller may be configured to detect potential confounding variables in real time. For example, the controller may track all variables of all humans in the sRCT emulation and determine that one or more variables as having a statistically significant impact on outcomes. In response to this determination, the controller may immediately flag this variable as a potential confounding variable to be gathered in all instances. Specifically, suppose a situation where the controller in one instant realizes that a variable that has been gathered for purposes other than the sRCT emulation for a number of other interactions is a potential confounding variable, and one millisecond later causes a prompt for a current ongoing interaction with a human to gather data on this variable. In this way, by gathering data from interactions and analyzing in real-time how this data impacts current results of the sRCT emulation, the controller may improve an ability to gather all data that is relevant for the sRCT emulation and ultimately improve the result of the sRCT emulation.

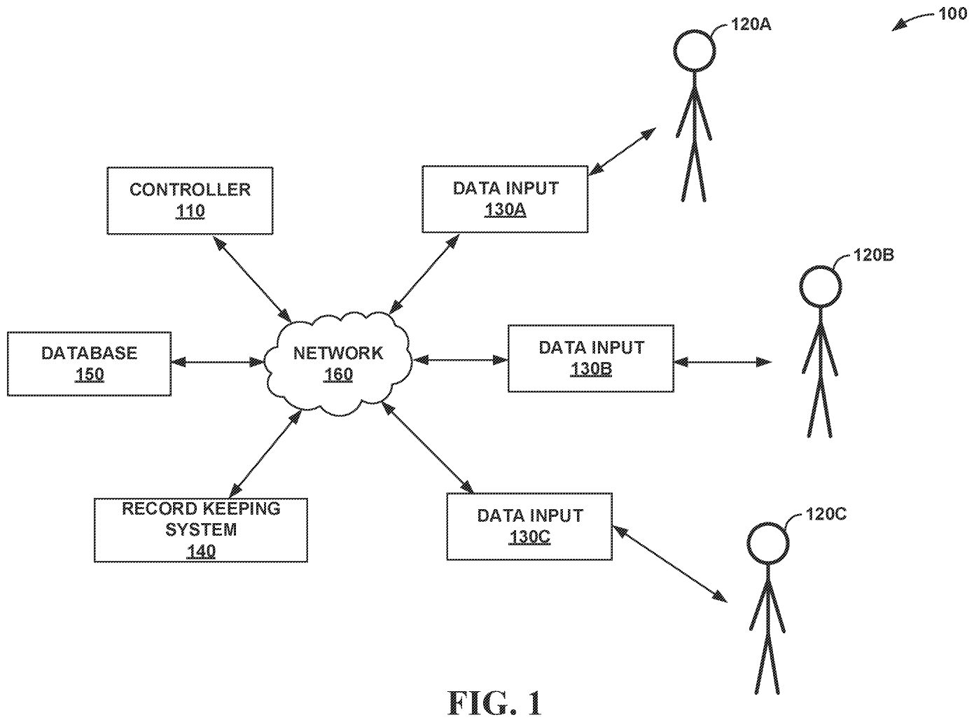

For example, FIG. 1 depicts environment 100 in which controller 110 manages data from a plurality of humans 120A-120C (collectively, “humans 120”) that are each in the middle of an interaction in which data is being gathered via a plurality of data inputs 130A-130C (collectively, “data inputs 130”). Humans 120 may be in these interactions for reasons that have nothing to do with any study. For example, the sRCT may be a medical study, and humans 120 may be in interactions that are unrelated medical appointments. For another example, the sRCT may relate to a financial study, and humans 120 may be in interactions that are financial appointments with bankers or investors or the like. In some examples these interactions (e.g., the medical appointments or meetings with a banker or investor) may be scheduled for reasons other than a sRCT, though in other examples, one or more of these interactions may have been scheduled for express purpose of the sRCT.

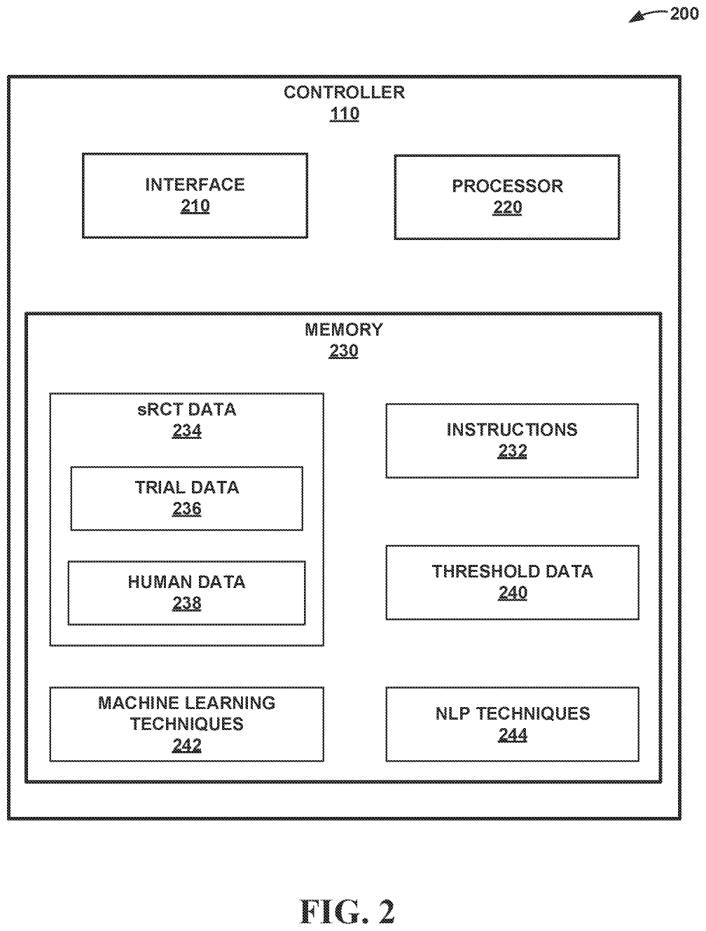

Controller 110 may include a computing device, such as computing system 200 of FIG. 2 that includes a processor communicatively coupled to a memory that includes instructions that, when executed by the processor, causes controller 110 to execute one or more operations described below. In some examples, controller 110 may be incorporated into (or be functionality of) record keeping system 140 that is used to store data of interactions, such as medical record keeping system 140 that is configured to store electronic health records (EHRs, otherwise known as electronic medical records). In other examples, controller 110 may be external to record keeping system 140, and may gather data in real-time as it travels between data inputs 130 and record keeping system 140. For example, data inputs 130 may be computing devices such as laptops or desktop computers, where these computing devices send data across network 160 to a hosted record keeping system 140 (e.g., hosted on a cloud and access on computing device data inputs 130). In other examples, data inputs 130 may be smart devices that gather data on humans, such as a scale (to measure weight) or an x-ray machine, or the like, such that these data inputs send these datapoints across network 160 to record keeping system 140. Controller 110 may intercept messages as they are sent across network 160 from data inputs 130 to record keeping system 140, and/or controller 110 may detect and gather these messages via agents at record keeping system 140 that forward data points of these messages to controller 110.

Network 160 may include one or more computer communication networks. An example network 160 can include the Internet, a local area network (LAN), a wide area network (WAN), a wireless network such as a wireless LAN (WLAN), or the like. Network 160 may comprise copper transmission cables, optical transmission fibers, wireless transmission, routers, firewalls, switches, gateway computers and/or edge servers. A network adapter card or network interface in each computing/processing device (e.g., controller 110, data inputs 130, record keeping system 140, and/or database 150) may receive messages (e.g., such as messages that include datapoints regarding humans 120 as gathered during interactions) and/or instructions from and/or through network 160 and forward the messages and/or instructions for storage or execution or the like to a respective memory or processor of the respective computing/processing device. Though network 160 is depicted as a single entity in FIG. 1 for purposes of illustration, in other examples network 160 may include a plurality of private and/or public networks over which controller 110 may gather and analyze data from human interactions for sRCTs as described herein.

Controller 110 may store gathered data in database 150. Database 150 may include structured data that is stored in a manner that is readily analyzable. For example, database 150 may include structured datapoints that are stored in a manner for which correlations and connections between different data points is maintained (e.g., dates at which various datapoints were gathered) in a manner that is consistent across much or all of database 150 so that controller 110 may “mine” database for information and/or correlation. For another example, database 150 may include tables where each table is one of humans 120, treatment decision points are stored in rows, confounding variables that influence the treatment decisions are stored as columns, or the like.

In some examples, controller 110 may also store structured text relating to humans 120 in database 150. Structured text, as defined herein, may include text for which syntactical, linguistic, and/or thematic relationships are known for and between some or all words of the text. Some or all of the structured text of corpus 150 may previously have been unstructured texts and may have been turned into a structured format by controller 110 using natural language processing (NLP) techniques as described herein. For example, the unstructured text may include written/typed/spoken notes that were received/gathered by data inputs 130 and sent over network 160 to record keeping system 140 (and were subsequently gathered by controller 110 over network 160 and/or at record keeping system 140). Controller 110 may store structured text in a format that is mineable and analyzable, such that natural language correlations between datapoints within structured text is maintained as stored within database 150. For example, the structured text may be stored in such a way that metadata describing the individual words of an unstructured text are maintained, where this metadata defines the meaning of each word both individually and within the context of the complete unstructured text received from data inputs 130. In some examples, controller 110 may extract datapoints from structure text and store these datapoints within tables of database 150 as described above. Though controller 110 is depicted as separate from database 150 in FIG. 1 for purposes of illustration, in some examples controller 110 and database 150 may be part of a single computing system (e.g., part of a single hosted system with record keeping system 140).

Controller 110 may continue gathering data from humans 120 until an objective of the sRCT is complete. In some examples, controller 110 may receive an objective when controller 110 received a request to execute a sRCT. For example, the objective may relate to solving for one or more variables, such that controller 110 continues to monitor humans 120 and gather datapoints on these humans 120 until controller 110 can provide equations or the like that quantify/control these variables with at least a threshold confidence score. In other examples the objective may relate to a certain duration, a certain number of humans 120, and/or a certain number of interactions. For example, the objective may be to gather data from interactions with at least 1,500 humans 120 over five years, where each human 120 needs to have at least three different interactions from which datapoints are gathered (e.g., to track a trajectory of events). Other examples of objectives as would be understood by one skilled in the art of sRCTs are also possible.

Once controller 110 has gathered sufficient data to execute the objective, controller 110 may notify a user. For example, controller 110 may inform a data scientist that database 150 is filled with enough data to likely be able to estimate effects with sufficient precision. In some examples, controller 110 may generate a report once the objective is complete. Controller 110 may generate a report such that it includes data on confounding variables, efficacy results for various fact patterns, outcomes for different subpopulations of the population of humans 120, or the like. In some examples, controller 110 may generate periodic reports during an sRCT (e.g., a new report every week), reports in response to new potential confounding variables being identified by controller 110, and/or reports when various milestones are hit (e.g., achieving a critical mass of humans 120 needed to have a statistically significant sRCT). Such reports that are generated by controller 110 prior to the sRCT being concluded may be sent to one or more data scientists to enable these data scientists to change any parameters of the sRCT as are defined for controller 110.

As described above, controller 110 may be part of a computing device that includes a processor configured to execute instructions stored on a memory to execute the techniques described herein. For example, FIG. 2 is a conceptual box diagram of such computing system 200 of controller 110. While controller 110 is depicted as a single entity (e.g., within a single housing) for the purposes of illustration, in other examples, controller 110 may include two or more discrete physical systems (e.g., within two or more discrete housings). Controller 110 may include interfaces 210, processor 220, and memory 230. Controller 110 may include any number or amount of interface(s) 210, processor(s) 220, and/or memory(s) 230.

Controller 110 may include components that enable controller 110 to communicate with (e.g., send data to and receive and utilize data transmitted by) devices that are external to controller 110. For example, controller 110 may include interface 210 that is configured to enable controller 110 and components within controller 110 (e.g., such as processor 220) to communicate with entities external to controller 110. Specifically, interface 210 may be configured to enable components of controller 110 to communicate with data inputs 130, record keeping system 140, database 150, or the like. Interface 210 may include one or more network interface cards, such as Ethernet cards and/or any other types of interface devices that can send and receive information. Various numbers of interfaces may be used to perform the described functions according to particular needs.

As discussed herein, controller 110 may be configured to gather datapoints regarding humans 120 from interactions for purposes of an sRCT. Controller 110 may utilize processor 220 to gather data regarding humans 120 for the sRCT. Processor 220 may include, for example, microprocessors, digital signal processors (DSPs), application specific integrated circuits (ASICs), field-programmable gate arrays (FPGAs), and/or equivalent discrete or integrated logic circuits. Two or more of processor 220 may be configured to work together to gather data on humans 120 accordingly.

Processor 220 may gather data from humans 120 during interactions for an sRCT according to instructions 232 stored on memory 230 of controller 110. Memory 230 may include a computer-readable storage medium or computer-readable storage device. In some examples, memory 230 may include one or more of a short-term memory or a long-term memory. Memory 230 may include, for example, random access memories (RAM), dynamic random-access memories (DRAM), static random-access memories (SRAM), magnetic hard discs, optical discs, floppy discs, flash memories, forms of electrically programmable memories (EPROM), electrically erasable and programmable memories (EEPROM), or the like. In some examples, processor 220 may gather, store, and analyze data regarding humans for sRCTs according to instructions 232 of one or more applications (e.g., software applications) stored in memory 230 of controller 110.

In addition to instructions 232, in some examples gathered or predetermined data or techniques or the like as used by processor 220 to gather, store, and analyze data on humans 120 for sRCTs as described herein may be stored within memory 230. For example, memory 230 may include information described above as controller 110 gathers from data inputs 130. For example, as depicted in FIG. 2, memory 230 may include sRCT data 234, which itself includes trial data 236 and human data 238. Trial data 236 may include data on the objective of the sRCT, confounding variables of the sRCT, or the like. Human data 238 may include datapoints that have been gathered that are relevant for the sRCT. For example, if the sRCT is a medical study, human data 238 may include biographical information of respective humans 130, medical conditions of the humans, treatment plans that respective humans 130 are on, and the like. In some examples, controller 110 may functionally store all of database 150 within memory 230, such that all of database 150 is included within human data 238 or the like.

Further, memory 230 may include threshold data 240. Threshold data 240 may include a threshold at which controller 110 is to include humans 120 within a given sRCTs. For example, threshold data 240 may include the factors that define the population desired for the sRCT, and/or the specific percentage of the population that is to be filled by various subpopulations, and the like. For example, if sRCT is a medical study, the population may include humans 120 that have a specific medical condition, where threshold data 240 includes thresholds on an allowable minimum and/or maximum percentage of humans 130 of the sRCT that are under the age of 30, between the ages of 30 and 50, and older than 50.

Threshold data 240 may also include data on when and how to suggest various events to fully quantify various causal event chains as per the sRCT. For example, if the sRCT is part of a medical study, threshold data 240 may include data on when to suggest a type of medical treatment plan for respective humans 120. This threshold data 240 may include various factors that indicate that a treatment plan may or may not be warranted for respective humans 120.

Memory 230 may further include machine learning techniques 242 that controller 110 may use to improve a gathering data on humans 120 for sRCTs as discussed herein over time. Machine learning techniques 242 can comprise algorithms or models that are generated by performing supervised, unsupervised, or semi-supervised training on a dataset, and subsequently applying the generated algorithm or model to determine when to gather and when not to gather data regarding humans 120, and when to suggest and/or not suggest various future events to capture causal event chains for the sRCT. For example, using machine learning techniques 242, controller 110 may update one or more thresholds saved in threshold data 240 to improve a process of gathering data from humans 120 to execute sRCTs.

Machine learning techniques 242 can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques. For example, machine learning techniques 242 can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), partial least squares regression (PLSR), principal component analysis (PCA), principal component regression (PCR), Sammon mapping, t-distributed stochastic neighbor embedding (t-SNE), bootstrap aggregating, ensemble averaging, gradient boosted decision tree (GBRT), gradient boosting machine (GBM), inductive bias algorithms, Q-learning, state-action-reward-state-action (SARSA), temporal difference (TD) learning, apriori algorithms, equivalence class transformation (ECLAT) algorithms, Gaussian process regression, gene expression programming, group method of data handling (GMDH), inductive logic programming, instance-based learning, logistic model trees, information fuzzy networks (IFN), hidden Markov models, Gaussian naïve Bayes, multinomial naïve Bayes, averaged one-dependence estimators (AODE), Bayesian network (BN), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning algorithms.

Memory 230 may further include NLP techniques 244. NLP techniques 244 can include, but are not limited to, semantic similarity, syntactic analysis, and ontological matching. For example, in some embodiments, processor 220 may be configured to analyze unstructured text data as gathered from data inputs 130 to determine semantic features (e.g., word meanings, repeated words, keywords, etc.) and/or syntactic features (e.g., word structure, location of semantic features in headings, title, etc.) of unstructured texts. Ontological matching could be used to map semantic and/or syntactic features to a particular concept. The concept can then be used to determine the topic of unstructured texts regarding humans 120 as captured at data inputs 130. In this same way, controller 110 may identify datapoints on humans 120 when it is received as unstructured text data at data inputs 130.

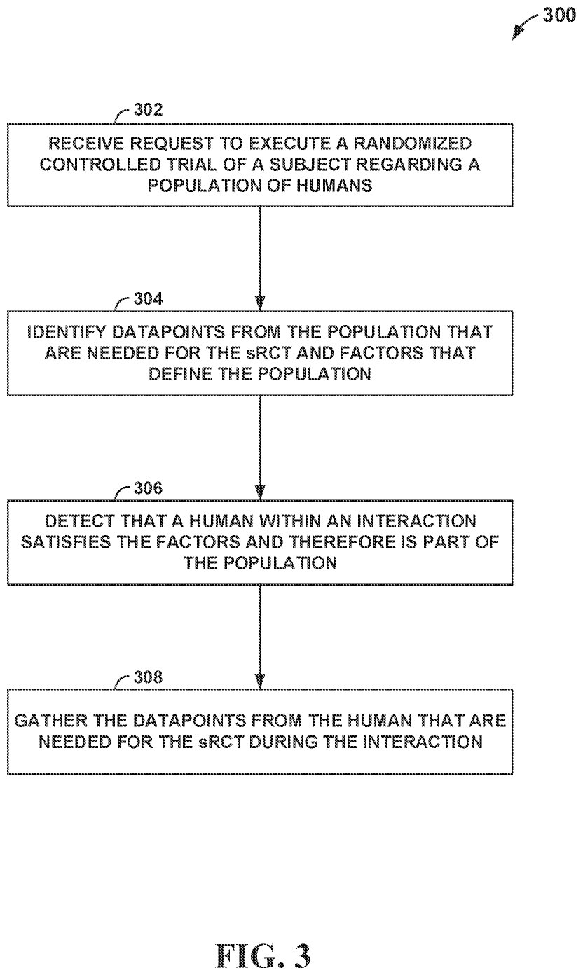

Using these components, controller 110 may gather data on humans 120 for sRCT studies as discussed herein. For example, controller 110 may gather data on humans 120 according to flowchart 300 depicted in FIG. 3. Flowchart 300 of FIG. 3 is discussed with relation to FIG. 1 for purposes of illustration, though it is to be understood that other systems and message may be used to execute flowchart 300 of FIG. 3 in other examples. Further, in some examples controller 110 may execute a different method than flowchart 300 of FIG. 3, or controller 110 may execute a similar method with more or less steps in a different order, or the like.

Flowchart 300 starts with controller 110 receiving a request to execute an sRCT or sRCT emulation for a subject that regards a certain population of humans 120 (302). A data scientist that is looked to conduct the sRCT may send the request to controller 110. The request as received by controller 110 may receive subsequent information for controller 110 to begin gathering data for the sRCT.

Controller 110 may identify datapoints that are needed from the population of humans 120 for the sRCT or sRCT emulation, and also identify factor that define the population as fitting the sRCT (304). In some examples the request for the sRCT may include parameters of the sRCT, including the specific subject of the sRCT, factors that define the population of the sRCT, and datapoints needed to complete the sRCT, such that controller 110 may identify datapoints that are needed and factors of the population from the request itself. In some examples, datapoints that are needed may change over time as controller 110 gathers more information, such that an analysis that controller 110 executes in identifying datapoints that are needed may change as controller 110 executes the data-gathering steps of the sRCT.

Controller 110 detects that one of humans within a respective interaction satisfies the factors and therefore is part of the population (306). For example, the sRCT may be a medical sRCT and the interaction is a medical appointment, and controller 110 may check factors stored within record keeping system 140 for the upcoming scheduled medical appointment, identifying that a respective human 120A of an upcoming medical appointment belongs to the population. In other examples, the interaction may be currently occurring (e.g., rather than scheduled for the future) and related to something other than the subject when controller 110 detects the interaction.

For example, the subject may be rosacea (a skin condition that causes redness on the face), whereas the interaction is a standard medical physical. In this example, during the standard medical physical, a medical professional may hear from human 120A of redness and visible blood vessels within face of human 120A, and may provide information along these lines to a desktop data input 130A of the medical professional. This information of the redness and visible blood vessels on face of human 120A may be written in natural language and sent in a message to record keeping system 140. Controller 110 may detect this message as it is sent to and/or arrives at record keeping system 140, and may use NLP techniques as described herein to detect “redness,” “visible blood vessels,” and “face” as data associated with human 120A. In some examples, controller 110 may determine from this first set of data that was gathered for the medical physical is sufficient to determine that human 120A belongs to the population as a result of having rosacea.

However, in other examples, controller 110 may determine that this first set of data that was gathered as part of the scheduled engagement of the medical physical is insufficient to determine whether or not human 120A belongs to the population. For example, controller 110 may determine redness and visible blood vessels as associated with a face of human 120A to be indications that human 120A might be part of the population. Controller 110 may therefore prompt the medical professional to gather more data from the human regarding the specific factors that indicate that humans 120 are part of the population. For example, controller 110 may prompt the medical professional to ask if any physical trauma proceeded the redness and visible blood vessels, and/or if there was a periodic nature to the redness and visible blood vessels. Controller 110 may then detect additional information regarding human 120A as received from data input 130A (as analyzed using NLP techniques) that human 120A did not have physical trauma, and also that the redness and visible blood vessels tend to come for weeks at a time. In response to this additional data received via the prompt, controller 110 determines that human 120A belongs to the population.

In some examples, controller 110 may detect that another human 120B has a future upcoming scheduled appointment for rosacea, such that human 120B satisfies the factors and therefore is part of the population. Controller 110 may then compare data of human 120B as stored within record keeping system 140, database 150, and the like against parameters for sRCT. For example, controller 110 may determine whether or not human 120B is part of a subset of the population that is already sufficiently represented in the sRCT. Specifically, controller 110 may determine that human 130B is a 35-year-old male, which is already at maximum representation within the sRCT. In response to this determination, controller 110 may determine to not gather datapoints needed for sRCT from human 120B into database 150. In some examples, controller 110 may flag human 120B as a possible participant and may gather information (and prompt medical professional to ask information), but may purposefully not use data of human 120B in reports as discussed herein in order to maintain statistically appropriate representation within population of database 150.

Controller 110 gathers the datapoints needed for the sRCT from respective humans 120 that are part of population during the interaction (308). This may include prompting medical professionals to ask humans 130 about the datapoints. This may also include gathering datapoints directly from various measuring devices, such as scales, magnetic resonance imagining (MRI) machines, X-rays, or any other device capable of capturing and electronically sending data of humans 120 across network 160.

Controller 110 may also detecting whether the interaction is relevant to the sRCT by virtue of a treatment decision point or outcome ascertainment point (e.g., a point at which one of a predetermined set of outcomes for a patient is ascertained) of the medical appointment. For example, controller 110 may check rules or an algorithm associated with the sRCT to see whether or not datapoints of the medical appointment match a treatment decision point and/or an outcome ascertainment point. If controller 110 determines, e.g., that the interaction matches factors relating to a treatment decision point, controller 110 randomly assigns a treatment that corresponds to this treatment decision point. Controller 110 may randomly assign the treatment with treatment probabilities determined conditionally on previously ascertained patient history.

In some examples, while receiving and analyzing data of humans 120 gathered during interactions, controller 110 may identify one or more confounding variables of the sRCT. Once controller 110 identifies such a confounding variable, controller 110 may prompt the medical professional to consider treatment plans as a result of the confounding variable. For example, confounding variables may indicate that different treatment plans have better relative efficacy compared to other portions of the population as a result of the confounding variable. Controller 110 might provide the confounding variable and suggest one or more treatment plans that corresponds to this confounding variable.

Controller 110 may identify a confounding variable of human 120 of an interaction, where this confounding variable was identified in the request that set up sRCT (e.g., such that a data scientist and/or medical professional identified this confounding variable for controller 110). In other examples, controller 110 may identify confounding variable as controller 110 analyzes all datapoints within database 150 for sRCT. For example, controller 110 may identify a variable that seems to be correlated with unexpectedly different results as compared to overlapping segments of human 120 population. In response to identifying a potential confounding variable, controller 110 may present this confounding variable to a data scientist and/or medical professional for analysis. In other examples, controller 110 may independently confirm that the identified factor is a confounding variable so long as the correlations have a sufficient confidence score.

In some example, controller 110 may suggest one or more future interactions with the respective human 120. For example, controller 110 may suggest follow-up medical appointments to gather subsequent data for the sRCT. Controller 110 may provide specific timelines for future medical appointments, and/or specific procedures to conduct during these medical appointments.

Controller 110 may detect that an amount of data gathered for the sRCT has reached a statistically significant threshold. For example, controller 110 may determine that data within database 150 is sufficient to support one or more conclusions with a statistical certainty. For another example, controller 110 may determine that one or more identified variables of the population have been found to have correlations with certain treatment plans and human 120 factors that satisfy a confidence score. In response to detecting that the amount of data gathered is statistically significant, controller 110 generates a report. This report may include all of some of the datapoints and statistical observations thereof. Controller 110 may provide this report and one or more corresponding conclusions to an appropriate party, such as a data scientist and/or medical professional.

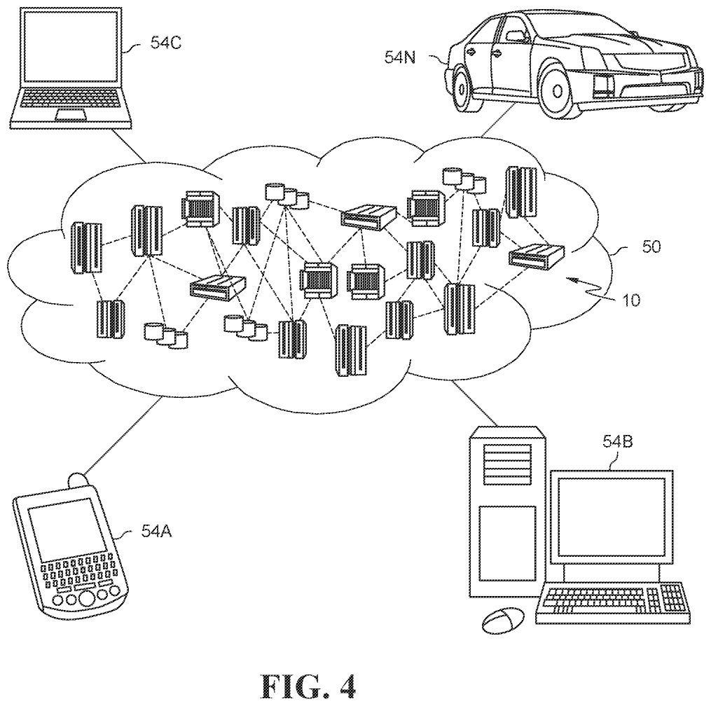

FIG. 4 illustrates an embodiment of a cloud environment, consistent with some embodiments. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain 3 resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

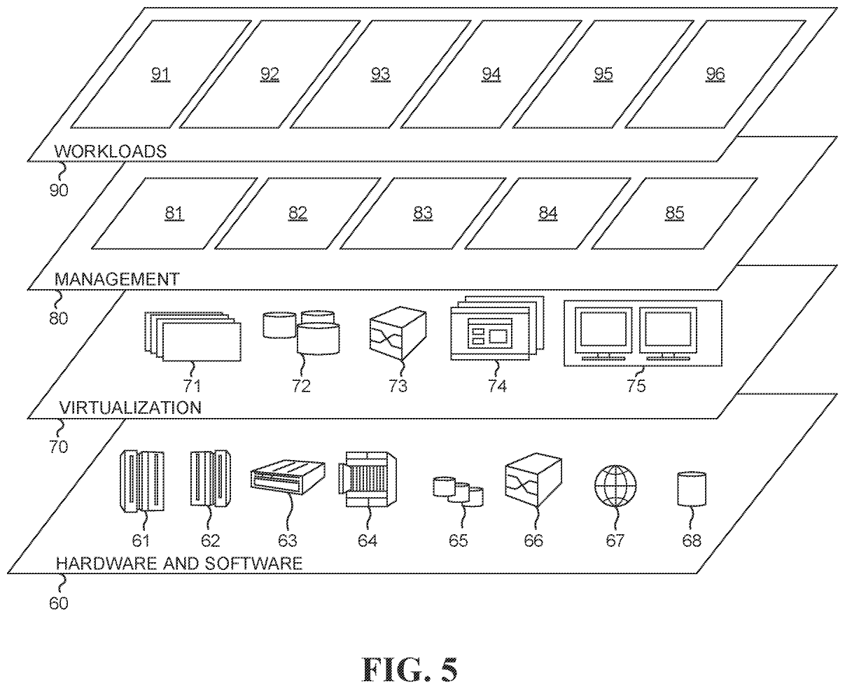

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and human 120 datapoint management during sRCTs 96.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-situation data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
receiving a request to execute a sequentially randomized controlled trial (sRCT) or sRCT emulation that relates to a subject regarding a population of humans;
identifying datapoints from the population that are needed for the sRCT or sRCT emulation and factors that define the population as fitting the sRCT;
detecting that a human within an interaction satisfies the factors and therefore is part of the population;
gathering, during the interaction, the datapoints from the human that are needed for the sRCT in response to detecting that the human is part of the population.

2. The computer-implemented method of claim 1, wherein the sRCT is a medical sRCT and the interaction is a medical appointment.

3. The computer-implemented method of claim 1, further comprising:
detecting whether the medical appointment is relevant to the sRCT by virtue of a treatment decision point or outcome ascertainment point of the medical appointment, wherein the gathering the datapoints from the human is in response to detecting that the interaction is relevant to the sRCT; and
randomly assigning a treatment if the interaction is detected to be a treatment decision point, with treatment probabilities determined conditional on previously ascertained patient history.

4. The computer-implemented method of claim 2, wherein gathering the datapoints from the human includes prompting a medical professional to gather the datapoints from the human.

5. The computer-implemented method of claim 4, wherein the interaction is currently occurring for a scheduled engagement that relates to something other than the subject.

6. The computer-implemented method of claim 2, further comprising scheduling one or more future medical appointments for the human to gather future datapoints that are needed for the sRCT.

7. The computer-implemented method of claim 2, further comprising:
gathering a first set of data during the medical appointment;
identifying a confounding variable of the sRCT among the first set of data; and
prompting the medical professional to consider treatment plans as a result of the confounding variable.

8. The computer-implemented method of claim 1, further comprising gathering a first set of data as part of a scheduled engagement that relates to something other than the subject, wherein information that indicates that the human satisfies the factors is included within the first set of data.

9. The computer-implemented method of claim 1, wherein the interaction is scheduled for a future point in time.

10. The computer-implemented method of claim 1, wherein the human is a first human and the interaction is a first interaction, further comprising:
detecting that a second human within a second interaction satisfies the factors and therefore is part of the population;
determining that the second human belongs to a subset of the population that is already sufficiently represented in the sRCT; and
determining to not gather the datapoints from the second human in response to determining that the second human belongs to the subset of the population that is already sufficiently represented in the sRCT.

11. The computer-implemented method of claim 1, further comprising:
gathering a first set of data during the interaction;
identifying, from the first set of data, an indication that the human might be part of the population; and
prompting, during the interaction, more data to be gathered from the human regarding the factors, wherein the detecting that the human within the interaction that the human is part of the population is in response to analyzing the more data.

12. A system comprising:
a processor; and
a memory in communication with the processor, the memory containing instructions that, when executed by the processor, cause the processor to:
receive a request to execute a sequentially randomized controlled trial (sRCT) that relates to a subject regarding a population of humans;
identify datapoints from the population that are needed for the sRCT and factors that define the population as fitting the sRCT;
detect that a human within an interaction satisfies the factors and therefore is part of the population; and
gather, during the interaction, the datapoints from the human that are needed for the sRCT in response to detecting that the human is part of the population.

13. The system of claim 12, wherein the sRCT is a medical sRCT and the interaction is a medical appointment that relates to something other than the subject.

14. The system of claim 13, the memory containing additional instructions that, when executed by the processor, cause the processor to:
detect whether the medical appointment is relevant to the sRCT by virtue of a treatment decision point or outcome ascertainment point of the medical appointment, wherein the gathering the datapoints from the human is in response to detecting that the interaction is relevant to the sRCT; and
randomly assign a treatment if the interaction is detected to be a treatment decision point, with treatment probabilities determined conditional on previously ascertained patient history.

15. The system of claim 13, wherein gathering the datapoints from the human includes prompting a medical professional to gather the datapoints from the human.

16. The system of claim 14, the memory containing additional instructions that, when executed by the processor, cause the processor to schedule one or more future medical appointments for the human to gather future datapoints that are needed for the sRCT.

17. The system of claim 12, wherein the interaction is currently occurring for a scheduled engagement that relates to something other than the subject, the memory containing additional instructions that, when executed by the processor, cause the processor to gather a first set of data as part of the scheduled engagement, wherein information that indicates that the human satisfies the factors is included within the first set of data.

18. The system of claim 12, the memory containing additional instructions that, when executed by the processor, cause the processor to:
detect that an amount of data gathered for the sRCT has reached a statistically significant threshold;
generate a report on the sRCT; and
provide the report and one or more corresponding conclusions.

19. A computer program product, the computer program product comprising a computer readable storage medium having program instructions embodied therewith, the program instructions executable by a computer to cause the computer to:
receive a request to execute a sequentially randomized controlled trial (sRCT) that relates to a subject regarding a population of humans;
identify datapoints from the population that are needed for the sRCT and factors that define the population as fitting the sRCT;
detect that a human within an interaction satisfies the factors and therefore is part of the population; and
gather, during the interaction the datapoints from the human that are needed for the sRCT in response to detecting that the human is part of the population.

20. The computer program product of claim 19, wherein the sRCT is a medical sRCT and the interaction is a medical appointment that relates to something other than the subject, the computer readable storage medium containing additional instructions that, when executed by the computer, cause the computer to:
detect whether the medical appointment is relevant to the sRCT by virtue of a treatment decision point or outcome ascertainment point of the medical appointment,
wherein the gathering the datapoints from the human includes prompting a medical professional to gather the datapoints from the human and is in response to detecting that the interaction is relevant to the sRCT;
randomly assigning a treatment if the interaction is detected to be a treatment decision point, with treatment probabilities determined conditional on previously ascertained patient history; and
schedule one or more future medical appointments for the human to gather future datapoints that are needed for the sRCT.
